(19) United States
(12) Patent Application Publication
(10) Pub. No.: US 2021/0327595 A1
(43) Pub. Date: Oct. 21, 2021

(54) SYSTEMS AND METHODS FOR TRACKING AND MANAGING INFECTIOUS DISEASES WHILE MAINTAINING PRIVACY, ANONYMITY AND CONFIDENTIALITY OF DATA

(71) Applicant: Mohammad Abdel-Fattah Abdallah, El Dorado Hills, CA (US)

(72) Inventor: Mohammad Abdel-Fattah Abdallah, El Dorado Hills, CA (US)

(21) Appl. No.: 17/234,675

(22) Filed: Apr. 19, 2021

Related U.S. Application Data
(60) Provisional application No. 63/012,009, filed on Apr. 17, 2020.

(51) Int. Cl. G16H 50/80; G16H 50/20; G16H 40/67; G06F 21/62; G06N 20/00; A61B 5/00

(52) U.S. Cl. CPC G16H 50/80; G16H 50/20; G16H 40/67; A61B 5/0077; G06N 20/00; A61B 5/7267; A61B 5/6898; G06F 21/6254

(57) ABSTRACT

Embodiments of the present invention provide an artificial intelligence-enabled apparatus, such as a mobile communication device equipped with input ports and bio sensors and executing controlling, coordinating, managing software algorithms for reducing the spread of a pandemic and for managing social and economic impacts to minimize disruptions that occur due to indiscriminate and global actions such as strict social distancing and business closures. User information is maintained private and secure, and can be used to detect and warn about potentially dangerous situations, such as a user being in close proximity to potentially infected individuals.

### CROSS-REFERENCE TO RELATED APPLICATIONS:

This application claims the benefit of and priority to provisional patent application Ser. No. 63/012,009, Attorney Docket Number ABDA-P001.PRO, entitled “System with Smart device, intelligent Apparatus and technology methods to predict, minimize, track and manage an infectious pandemic while maintaining privacy and confidentiality of individuals,” with filing date Apr. 17, 2020, which is hereby incorporated by reference in their entirety as if fully set forth below.

### FIELD

Embodiments of the present invention generally relate to the field of information technology. More specifically, embodiments of the present invention relate to systems and methods for tracking and communicating information securely and confidentially.

### BACKGROUND

Pandemics pose great threat to human life on the health, social, educational and economic levels. One great problem is that individuals in a pandemic might be spreading the illness without others knowing or even they themselves may not be aware of it. This leads to one of two undesired outcomes to manage the problem on different levels, e.g., on the individual level, the business/education entities level and the government level.

One traditionally used approach is to keep life, social, education and business disruptions at low levels while relying on treating cases at health facilities when they become ill. This approach in a pandemic scenario may lead to massive spreading of the illness and an inability of the health system to handle the large illness numbers in addition to an inability to contain the spread among new individuals. This approach is usually taken because a particular society is unaware of the real underlying early spread of the illness that can go undetected and also because no real time efficient mechanism exists to backtrack and trace new cases and to uncover people's potential past infectious history as they were mixing with other individuals.

The other approach is to implement a strict quarantine, stay at home, or even curfew. Such severe measures are indiscriminate and in the long run cause major disruption to life, social, education activities which can cause great harm to the economy.

### SUMMARY

Embodiments of the present invention provide an artificial intelligence-enabled apparatus, such as a mobile communication device equipped with input ports and bio sensors and executing, controlling, coordinating, and managing algorithms for reducing the spread of a pandemic and managing social and economic impacts to minimize disruptions that occur due to indiscriminate and global actions such as strict social distancing and business closures. With the novel approach, user information is maintained private and secure, and can be used to detect and warn about potentially dangerous situations, such as proximity to potentially infected individuals.

According to one embodiment, a method of securely tracking potential infections of a contagious disease is disclosed. The method includes verifying an identity of a first user using a first sensor of a mobile computer device, capturing a biometric feature of the first user using the first sensor of the mobile computer device, training an artificial intelligence machine learning apparatus using baseline symptoms associated with a contagious disease, and the AI machine learning apparatus predicting a likelihood of potential infection of the contagious disease of the first user according to the biometric feature.

According to another embodiment, a system for securely tracking potential exposure to a contagious disease is disclosed. The system includes an artificial intelligence machine learning apparatus, and a first smart device operable to engage in communication with the AI machine learning apparatus and further operable to execute a software application that performs a first method comprising verifying an identity of a first user using a first sensor of the first smart device, and capturing and storing a biometric feature of the first user using the first sensor of the first smart device. The AI machine learning apparatus is operable to execute a software application that performs a second method comprising performing machine learning using baseline symptoms associated with a contagious disease, and predicting a likelihood of potential infection of the contagious disease of the first user according to the biometric feature.

According to a different embodiment, a smart device is disclosed. The smart device includes a finger scanner input device, a camera input device, an audio input device, and a processor in electronic communication with the finger scanner input device, the camera input device, and the audio input device. The processor is operable to execute instructions to automatically perform a method including authenticating a user using at least one of the input devices, capturing biometric data of the finger scanner input device, the camera input device, and the audio input device, transmitting biometric data anonymously to an artificial intelligence (AI) machine learning apparatus, and receiving a predicted outcome for the user from the AI machine learning apparatus generated according to the biometric data.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 depicts an exemplary table of private user data for Individual A, including time, location, proximity devices, and sensor data for tracking, tracing, and predicting infections according to embodiments of the present invention.

FIG. 2A depicts an exemplary table of private user data for Individual B, including time, location, proximity devices, and sensor data for tracking, tracing, and predicting infections according to embodiments of the present invention.

FIG. 2B depicts an exemplary table of private user data for Individual B, including time, location, proximity devices, and sensor data for tracking, tracing, and predicting infections according to embodiments of the present invention.

FIG. 3 depicts an exemplary computer system including a central AI component and multiple mobile devices in communication over a wireless network for tracking, tracing, and predicting infections according to embodiments of the present invention.

FIG. 4 depicts exemplary electronic devices including input ports that capture image data, audio data, etc., for tracking, tracing, and predicting infections according to embodiments of the present invention.

FIG. 5 depicts an exemplary sequence of computer implemented steps for extracting samples of biometric features for AI training, infection prediction, and back-tracking according to embodiments of the present invention.

FIG. 6 depicts an exemplary sequence of computer implemented steps for extracting samples of biometric features for AI training, infection prediction, and policy making according to embodiments of the present invention.

### DETAILED DESCRIPTION

Reference will now be made in detail to several embodiments. While the subject matter will be described in conjunction with the alternative embodiments, it will be understood that they are not intended to limit the claimed subject matter to these embodiments. On the contrary, the claimed subject matter is intended to cover alternative, modifications, and equivalents, which may be included within the spirit and scope of the claimed subject matter as defined by the appended claims.

Furthermore, in the following detailed description, numerous specific details are set forth in order to provide a thorough understanding of the claimed subject matter. However, it will be recognized by one skilled in the art that embodiments may be practiced without these specific details or with equivalents thereof. In other instances, well-known methods, procedures, components, and circuits have not been described in detail as not to unnecessarily obscure aspects and features of the subject matter.

Reference will now be made in detail to several embodiments. While the subject matter will be described in conjunction with the alternative embodiments, it will be understood that they are not intended to limit the claimed subject matter to these embodiments. On the contrary, the claimed subject matter is intended to cover alternative, modifications, and equivalents, which may be included within the spirit and scope of the claimed subject matter as defined by the appended claims.

Furthermore, in the following detailed description, numerous specific details are set forth in order to provide a thorough understanding of the claimed subject matter. However, it will be recognized by one skilled in the art that embodiments may be practiced without these specific details or with equivalents thereof. In other instances, well-known methods, procedures, components, and circuits have not been described in detail as not to unnecessarily obscure aspects and features of the subject matter.

Portions of the detailed description that follows are presented and discussed in terms of a method. Although steps and sequencing thereof are disclosed in a figure herein describing the operations of this method, such steps and sequencing are exemplary. Embodiments are well suited to performing various other steps or variations of the steps recited in the flowchart of the figure herein, and in a sequence other than that depicted and described herein.

Some portions of the detailed description are presented in terms of procedures, steps, logic blocks, processing, and other symbolic representations of operations on data bits that can be performed on computer memory. These descriptions and representations are the means used by those skilled in the data processing arts to most effectively convey the substance of their work to others skilled in the art. A procedure, computer-executed step, logic block, process, etc., is here, and generally, conceived to be a self-consistent sequence of steps or instructions leading to a desired result. The steps are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussions, it is appreciated that throughout, discussions utilizing terms such as “accessing,” “writing,” “including,” “storing,” “transmitting,” “associating,” “identifying,” “encoding,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Some embodiments may be described in the general context of computer-executable instructions, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

### Systems and Methods for Tracking and Managing Infectious Diseases While Maintaining Privacy, Anonymity and Confidentiality of Data

Embodiments of the present invention provide an artificial intelligence-enabled apparatus, such as a mobile communication device equipped with input ports and bio sensors and executing controlling, coordinating, managing software algorithms for reducing the spread of a pandemic and for managing social and economic impacts to minimize disruptions that occur due to indiscriminate and global actions such as strict social distancing and business closures. User information is maintained private and secure, and can be used to detect and warn about potentially dangerous situations, such as a user being in close proximity to potentially infected individuals.

Some embodiments include an artificial intelligence-enabled apparatus, (e.g., a mobile communication device) equipped with input ports and biometric (“bio”) sensors having controlling/coordinating/managing methods, and software applications executing thereon that can reduce the spread of illness from the pandemic and manage the social and economic impacts to minimize disruptions that occur due to the indiscriminate and global actions such as strict social distancing and business closures. The system, device, apparatus, and method described herein can use medical diagnosis based on medical personnel judgment especially in absence of available precise tests, either early on in the pandemic where tests are not yet developed or because not enough testing capacity is available or because of logistics that prevent large number of people to be tested.

Some embodiments provide a system including individual electronic devices such as wearables or mobile communication devices (e.g., mobile smart phones, smart wearables) with additional hardware sensing components that combine the use of device input ports (such as image/video capture, audio capture, touch capture, etc.) with uniquely identifiable individual feature sensors alongside biometrics sensors in conjunction with a logically central (and potentially physically distributed) apparatus that uses artificial intelligence (AI)/machine learning hardware and software algorithms to perform training and analysis/prediction of illness and infectious status using a managing method and application to form a new system, and uses new methods to train and predict infectious status to help facilitate actions such as entry passes, boarding a mass transit, class attendance permit or entry through country border. Embodiments also enable the private recording of history of space and time records that are searchable using the central (or distributed) apparatus to identify, track and trace potentially infected/infectious individuals who were collocated (in time and space) in the past.

The tracking and tracing of past colocation history can be performed when the infectious status of an individual is known to be unsafe or predicted by the AI apparatus as all the records can identify the people who were collocated with the tracked individual in the past, which is beneficial if the devices and the managing application are implemented on a wide-spread basis. A mobile smart phone with those capabilities can be combined with additional bio sensors and new/modified uses of traditional sensors (e.g., image, video, audio, touch) using a managing application connected to an AI/machine learning (ML) central prediction, recording and tracking/tracing apparatus (e.g., hardware and software server infrastructure of distributed processing elements, such as a server farm, computing cloud, etc.). These functions can be carried out with privacy, confidentiality and anonymity, and the individual participating with his device in this wide scale system can be notified with his predicted infectious status. The individual can receive a notification that he or she was collocated with another individual who is confirmed or predicted to be infected.

According to some embodiments, an individual can privately obtain safe passes for entering events or participate in gatherings, mass transit, crossing borders, etc., based on his own predicted status which his device can obtain from the central apparatus. If he has a potential non-safe status, then the individual can self-quarantine (which can be potentially verified by the device location history during the quarantine time and proximity detection of other individuals), or receive formal medical testing or further evaluation. Then his status can change on his device to potentially safe status. All predicted safe/unsafe status is not a guarantee of such status; however, embodiments can mitigate and reduce illness, but cannot completely eliminate risk or guarantee any safe or unsafe status because some infected individuals have undetected symptoms who may remain unidentified even with the use of AI systems. Other preventive and protective measures such using masks, cleaning and washing hands and not touching one's face, mouth, nose, etc. is still required, as well as keeping enough distance from each other in any colocation situation.

Embodiments of the present invention provide a system including personal mobile devices with enhanced/modified input ports and bio sensing capabilities, connected within a network (e.g., a wireless network) to central hardware apparatus and a method wherein an early detection mechanism uses real time hardware sensors and input ports implemented in a personal mobile device to collect individual-identified sensed data that combines both bio metrics with modified-use traditional input ports (e.g., image, video, audio and touch) as well as capturing individual identification and/or identity recognition. The device and sensors work in coordination with a managing or supervising method implemented using the device, an apparatus, and a managing application to acquire additional information for self-reporting symptoms. The personal mobile devices with the input ports, sensors, and software application are connected to a logically centralized (but potentially physically distributed) apparatus that employ artificial intelligence machine learning schemes to train its machine learning algorithms using large amounts of biological data collected through the device and system and to analyze, detect and eventually predict potential infections (“cases”) that can be then identified as a candidate for formal infection medical testing and medical attention.

Personal smart mobile devices/wearables can be used to continuously monitor and collect large scale authenticated and privately and anonymously concealed bio data as a means to predict infectious safe status described previously. As some of the those individuals eventually get confirmed to be infectious by way of medical resolution (e.g., formal testing), the system continuously receives feedback for AI training, and keeps improving its capabilities using the large biological database that is being collected continuously and the feedback that is obtained when some of those individuals receive a positive test result. The use of this trained system including continuously bio sensing and collecting data from those personal mobile devices is advantageous compared to traditional ways of having to formally diagnose the cases through physical testing or medical evaluation, because of the presence of huge data sets acquired as described herein and through use of traditional input ports (e.g., image/video, audio, and touch) to extract biological characteristics and features, and to use multiple bio sensing devices to jointly train the machine learning prediction. Because the monitoring and sensing and analysis of those individuals are continuously monitored and collected, the machine learning hardware, its machine learning models, and its running algorithms will become better at inferring and predicting future infectious status.

In contrast to existing systems that monitor and collect biological data, embodiments of the present invention can continuously collect data from bio sensors embodied in a personal mobile device that is continuously collecting the bio data, and the massive collection of this data can be enabled by a connected system that combines the different types of bio sensing data (e.g., sensors of a smart device) and identity recognition data fed into a machine learning apparatus that is then used to enable social, education, business and authority actions by predicting potential safe status of individuals.

According to some embodiments, the system employs a method to back track and trace the infection history of those predicted by the system (or using the status of positively tested identified individuals) to uncover all other individuals who could have potentially contacted the infection from those identified individuals. This approach can reduce unidentified community spread cases by backtracking in time and space to identify individuals who could potentially be infected as a result of temporal and spatial sharing (e.g., sharing place circles within certain time frames). Embodiments further provide an efficient way for education entities, businesses and authorities to create a safe pass/board decision by combining the apparatus AI predicted outcome obtained from each individual authenticated device, including sensed bio data, apparatus recorded history tracking and tracing of the device user, etc.

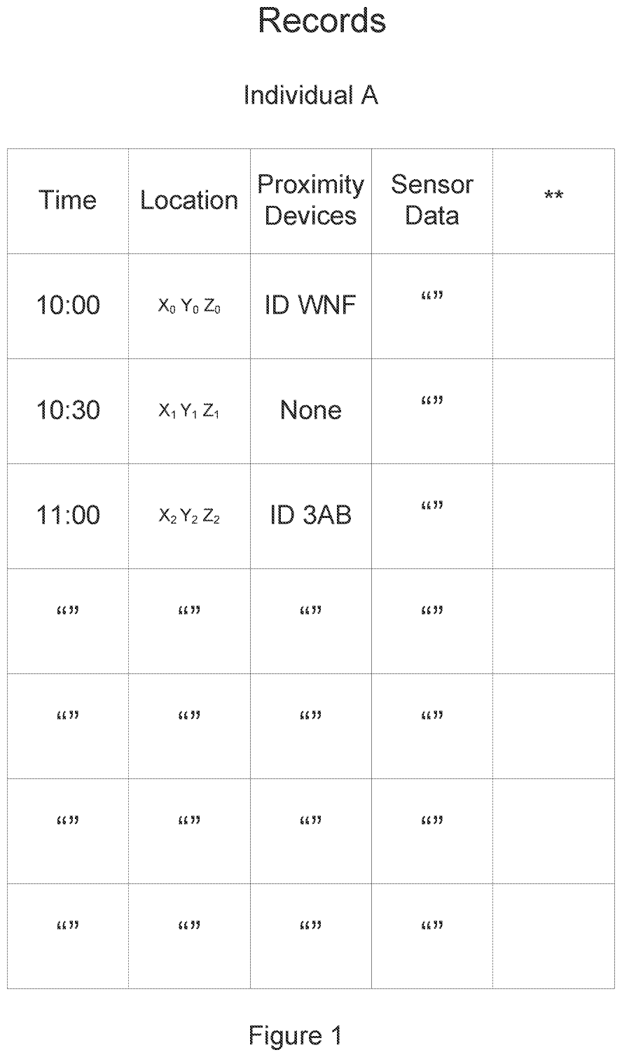

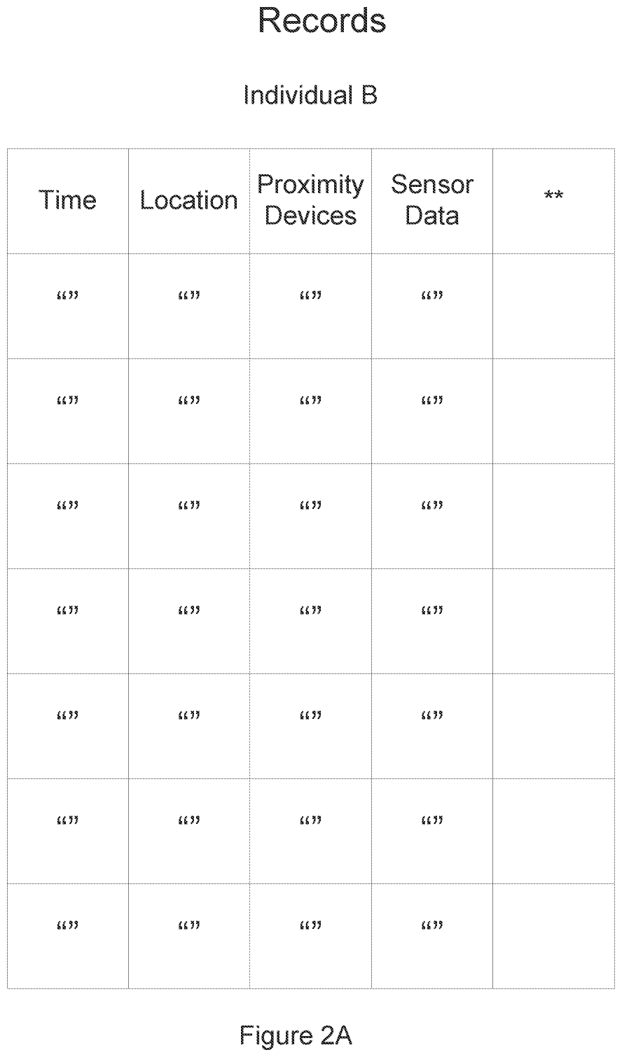

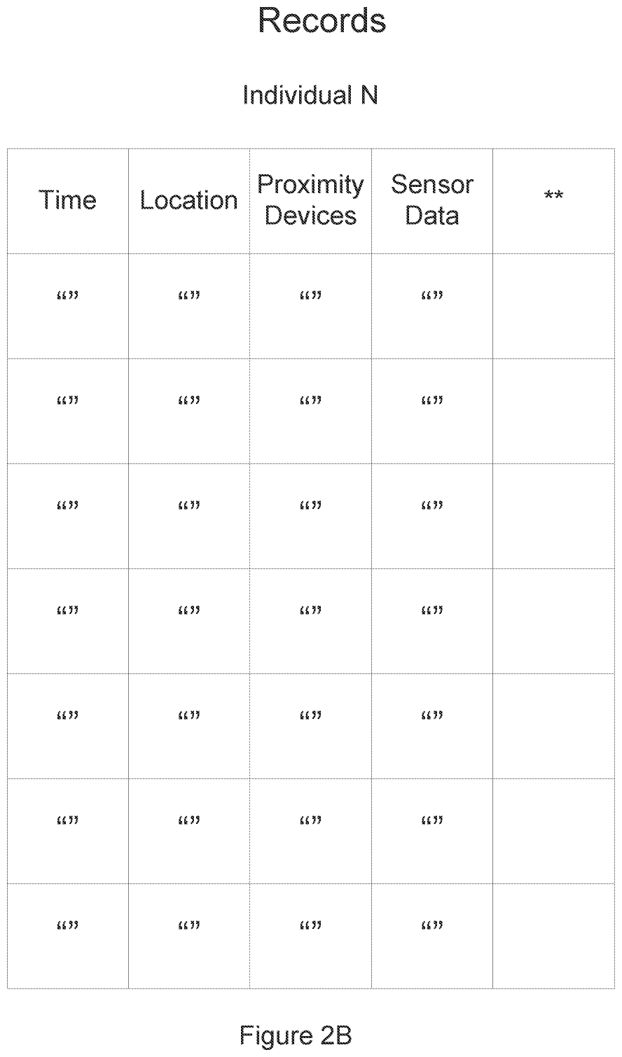

FIGS. 1-3 depict exemplary tracking computer records of users for tracking, tracing, and predicting infections according to embodiments of the present invention. In FIG. 1, the records of Individual A are kept private and anonymous. The records include a time associated with a location, the location (e.g., coordinate data), devices in proximity to the individual's devices at the time, and any sensor data captured at the time. In FIG. 2A, the records of Individual B are kept private and anonymous. The records include a time associated with a location, the location (e.g., coordinate data), devices in proximity to the individual's devices at the time, and any sensor data captured at the time. In FIG. 2B, the records of Individual C are kept private and anonymous. The records include a time associated with a location, the location (e.g., coordinate data), devices in proximity to the individual's devices at the time, and any sensor data captured at the time. According to some embodiment, each individual history is securely stored in memory by the individual's personal mobile device and is available to the system as anonymous data points. The recorded data is only used when the person chooses to use it in order to obtain access or an entry permit during a pandemic, or when the authorities request history back tracking and tracing to avoid imminent danger to other individuals when a person is confirmed as infectious so that all others who were collocated with him in space and time are alerted.

Other metrics such as electronically linked test results, vaccine status, attendance permits, safe access rights to a business perimeter, a boarding pass for mass transit or an entry authorization (e.g., a pass into country or state boarders) can be updated in real-time. Although those actions can be monitored by controllers of the entity executing the system and the machine learning algorithms, the system can alternatively use automated machine readable barcodes that can be scanned from the mobile device screen upon an attempt to access any of the mentioned activities, or it can also be used by the individual to determine that he is being safe to others if he were to conduct such actions. The predicted potentially non-safe status that is signaled by the apparatus through the device could also indicate to the individual that he might need to seek formal medical testing to help him avoid the consequences of actually getting sick from the potential infection.

### Novel Sensing Techniques for Predicting or Detecting Infections Diseases

According to some embodiments, a mobile or wearable device is provided that can connect with other mobile devices and other apparatus through local, proximity, and wireless communication protocols and networks using novel methods to use input ports and sensing capabilities. In one exemplary method, the characteristics of a human eye (and/or face) are captured using video or photo received via an input port of the mobile device of the user (the “device holder”). In another example, the temperature of a human body can be measured to detect the presence of a fever. This measurement can be performed by touching the device surface (e.g., a screen or its thumbprint modified sensor capable of measuring fever like a thermostat).

Other sensing capabilities can be implemented using audio of breathing sounds captured by the mobile device holder using a microphone embedded in the device. Other biometrics can be obtained using traditional bio sensing devices found in such mobile device such as heart/pulse rate sensors, blood pressure and other such biometrics sensors. Although some of the data is using captured using standard input ports capable of capturing image, video, audio, touch, etc., they can all be used to create and analyze a bio-informational state and establish a continuously evolving bio-informational state of the individual by comparing the bio analyzed data with historical data and comparing it to other individual information to allow an AI machine learning apparatus to predict potential infection status. Although the image/video, audio, and/or touch data captured by the sensors is collected for the specific user, other than characteristics and features extracted and parameters that are used by the artificial intelligence and machine learning algorithms, none of the data is transferred to the central apparatus in its original format, nor is it stored in its original format, in order to ensure the privacy of the device holder image, video, audio and fingerprint, etc. For instance, the data can be encoded.

All the accumulated device sensed data characteristics and feature extractions will be compared against a trained database through artificial intelligence apparatus with topologies such as neural networks using training and inference hardware and algorithms. For example, at the server side/cloud of the network and potentially also using device AI feature extraction and also inference support, such device-obtained data and apparatus artificial intelligence hardware and algorithms are combined to train the apparatus AI prediction which will employ a set of methods and machine learning algorithms to come at an infection prediction, and then notify of individual need for testing and also using vaccine status and prior infection status as an input and also using the central apparatus with such collected data to identify backtracking and tracing status and to employ an apparatus managing application to notify and indicate actions (such as testing recommendation and a potential predicted safe pass status) that can be used to indicate potential to attend/access group events as in class attendance, entrance permit to business/retail complexes, boarding passes for mass transits or entry pass through state borders.

According to some embodiments, the bio-sensed data that is analyzed can be automatically linked to the identity of the mobile device holder. For example video or a photo of the eyes or other portions of the device holder's face can be used to verify the identity of the individual being analyzed through a face or eye recognition scheme in addition to being used to detect possible underlying illness/infection or health issues (e.g., detecting eye color changes, texture changes, humidity, shape, dehydration, paleness, etc.). The same method can be used to measure the temperature of the device holder using the touch of his finger/hand while at the same time confirming his identity using the fingerprint image to read his temperature for identifying the presence of a fever. This creates a trusted environment such that this scheme and underlying device can be used by the individual himself or anonymously without explicitly publicizing the name and identity of the device holder.

In one example, a mobile device executes a software application to continuously collect the verified bio sensed data and input collected features of the individual device holder, and the device through uses the software application to continuously communicate the collected data to a remote apparatus which uses the data through as input to AI machine learning hardware and algorithms that predict non-safe (infected diagnosis) status. The apparatus can also use its history of collected data to determine the location and proximity of others who may have been collocated with this individual in the past, and their status can be changed to be infectious by formal medical testing or as a result of an AI apparatus predication based on the individuals own collected bio data. When the state of an individual becomes potentially infected, then the apparatus communicates to the device and to the holder that his safe status has changed to non-safe so that he or she can get formal medical testing and diagnosis. The user can also put himself or herself in quarantine and avoid any gathering or group educational activities, or avoid entering business premises, boarding mass transits, or crossing a boarder from state to state, etc.

Depending on privacy laws, educational entities, businesses, authorities can use the status displayed by an individual's own device (with the individual's consent consent) and without collecting/obtaining the individual's actual personal identity for granting actions like entering education or business premises, attending public or private gatherings, crossing borders, and similar activities. This can be done without revealing the identity of the person but rather by simple scanning the device holder's safe pass (similar to a boarding-pass) presented by the device screen using a displayed barcode or equivalent. The safe pass can be verified according to the device holder's thumbprint or face recognition features captured by his device to instantly authenticate his safe pass status. This type of authentication allows the application on the device to verify that the bio data was collected from the same person that is currently holding the device using a combination of bio metrics and identity recognition. According to some embodiments, the form of identity (e.g., fingerprint or face image) used for identity and bio data used for authentication is only stored by the user device application. This information is not stored and linked together at the central apparatus side; instead, the device generates a unique self-verified token, such as a special sequence number unique to the device and verifiable by the device which generated it to link all data and predicted status communications between the device and central apparatus. It is important to note that the identification of the device holder does not mean that his identity is known to others, but rather that the bio collected data (which is used to predict the potential safe status) is associated with the same person who is attempting to obtain a safe pass. Otherwise, the device holder will be automatically notified by his device that he may need to seek formal medical testing/diagnoses or care. The individual personal identity (e.g., name, address, phone number, thumbprint, face image) are all concealed and never shared from the user device to any other device or central apparatus without the device holder's consent. According to some embodiments, the system includes 3D location sensing and close-proximity location identifiers. The 3D location sensing can be performed using enhanced 3D global positioning system (GPS) scheme provided by mobile devices. The proximity sensing/identifying can be provided using schemes such as Bluetooth.

Some embodiments of the present invention provide a managing entity, algorithm and/or application that can run on a mobile device to collect the sensed data described above, and to communicate with a backend server at the network or cloud side. In one example, a managing entity application is used by businesses or education entities to anonymously identify individuals associated with a potential hazard (e.g., infection) where the identity is confirmed and authenticated but not revealed. Further, the health risks are sensed, analyzed and predicted by the apparatus, so the application running on the mobile device and the overall system can timely identify and evaluate in real-time the existence of a potential health hazard. This information can be used by the businesses, education entities, retailers, office complexes, etc., to grant or deny access. Furthermore, authorities at airports or other entry points can use the outcome results and utilize applications running on individual's mobile devices linked to the overall system in real time to grant entry to the country, state or city, and to allow individuals to board an airplane, bus or train or any other mass transit medium. Individuals who can obtain further testing to confirm the analytical assessment that was carried out by the artificial intelligence scheme using the sensor information. It is worth mentioning that the artificial intelligence scheme employs training data collected from analysis that is either confirmed or otherwise becomes a false positive to then form a feedback loop for artificial intelligence training.

Another important element of the embodiments of the present invention are the tracking and tracing of potential contamination to other individuals who later are confirmed to be infected carriers of an infection in accordance with 3D location or enhanced GPS data, as well as proximity sensing mechanisms using near-field communication like Bluetooth and other such mechanisms. When an individual is later confirmed to be infected, the data logged by the application regarding the real time date for the recorded 3D location and time frame as well as proximity data and time frame are used to search the secured database for other individuals who were either in the surrounding area of that person within a certain time frame or within a proximity of that individual in the past at certain times. These individuals can be notified privately via their own devices regarding their potential infection, and their device status can be automatically changed to potentially not safe. This is achieved by logging in a database using the managing application and the apparatus backend storage in the cloud. For example, the timestamp of all locations traversed by each device within a time frame (e.g., within an incubation period of an infection) can be accompanied by a log of all other devices (belonging to other individuals) where the proximity sensor was able to detect the other device or device.

Using a nearest search algorithm across all device holders and application users within the specific time frame (e.g., incubation timeframe) can identify all individuals who visited the same location or were near the location. Multiple circles or ranges of proximity and timeframes can be set to identify potential individuals at risk who were close by to this person or place and within certain timeframes. The variability in the time and place searches is needed because some infecting agents can stay active in locations and on surfaces long after the infecting individual has left the place. The search of the proximity log can also be performed on entries of other device holders who were close enough to register a Bluetooth synch between the different devices. This log can be stored long enough to cover at least the incubation period of the infection.

Using traditional approaches to infection tracking and notification, even after people are asked to stay home, many cases of light symptoms can go undetected for a long time. Accordingly, embodiments of the present invention can quickly identify individuals who may need to be tested to confirm infection using the device and managing application features of sensing, self-reported symptoms entry, and analysis of data with the help of AI prediction, as well as backtracking and tracing anyone in the past who was physically located at the same place within a certain period, or were within close proximity of the infected individual at a certain time. A software application executed by a user device (e.g., smartphone) is connected to a trusted environment and can be authenticated. Formal medical testing results can be used as training data for future refined artificial intelligence inference results, and to help businesses and authorities detect hidden confirmed cases to backtrack known cases. Information regarding the vaccination status of a device holder can be digitally linked to the device account/managing method and application to aid in the safe pass recommendation.

According to some embodiments, location data (e.g., GPS coordinates) can be tagged with additional information that provides further identification mechanisms for possible infectious history to try to identify collocated individuals at same place and time. The additional information can be collected from a mobile device over Wi-Fi, for example, to help track people who connected to the same Wi-Fi network at the same time. This approach helps provide addition information relating to the GPS location. Wi-Fi and other short distance communication protocols require close proximity so they can be used in addition to or in replacement of GPS location identification to detect proximity. This information can be memory stored locally on each device associated with the tracing and tracking system.

According to some embodiments, depth sensors can be used alongside camera or video capture to feed visually enhanced image characteristics and information to an AI machine learning apparatus for training, analysis, and prediction of each individual infectious state. Other than eye and face image/video capture and analysis, the camera can be further used to take images of a face that include the inner part of the mouth as well as the throat to be analyzed by AI machine learning to aid in diagnosis of infection. A microphone of the mobile device can be placed on the chest or back of the device holder while he or she takes a deep breath, and the recorded audio can be fed into the machine learning algorithm to train the algorithm and predict potential illness and the potential infectious category of the illness.

According to some embodiments, a smart watch, smart band, or smart earpiece can be used to collected bio metrics such as pulse rate, blood pressure, oxygen level, body temperature, and ear sound echo, for instance, which can be monitored, recorded, and fed into the AI machine learning apparatus to train AI/machine learning using biological data to analyze and predict infection or illness status. Moreover, bio-sensed data can be continuously or frequently captured and recorded through the input ports of a smart device (e.g., image, video, audio and touch, etc.), as well as bio metric sensors that measure body temp, pulse rate, blood pressure, oxygen level, etc. For example, when a fingerprint is typically used to unlock a smart phone by touching a screen or a button, a modified touch port (e.g., screen or button) can be used to capture the body/finger temperature and provide it as input to an AI/machine Learning apparatus and system.

When a face recognition image is used to unlock the smart phone, the image characteristics of the device holder's face and eyes (e.g., thinness, paleness, color and texture/hydration changes and other bio metrics of the eye or face) can be captured/recorded for analysis and training of the AI/ML apparatus and system. Similarly when the device holder's voice is used to unlock the phone or issue voice commands for the phone to perform actions, the audio characteristics (e.g., hoarseness, shortness of breath, etc.) can be captured and recorded for analysis and training of the AI/ML apparatus and system. Because these actions happen frequently and do not depend on the individual making an effort to do any bio metric collection, and the bio metrics collection does not depend on the individual feeling sick, the data will be continuously and frequently analyzed by the AI apparatus and used to train the machine learning algorithm to detect the patterns, changes, and characteristics obtained from the relatively large and frequent and biological data collection to achieve high prediction accuracy from the machine learning apparatus.

In contrast to traditional approaches to disease testing and tracking, embodiments of the present invention maintain a reasonable history record of bio sensed data about each of the individuals when the personal mobile device is used continuously. This allows the system and AI machine learning to recognize and settle differences and bio patterns in the metrics monitored, and therefore the state of each individual is unique to him. For example, a combination of a thumbprint used to unlock the device, the temperature of the device holder, the face recognition used to unlock the mobile device, and other bio metrics, such as breathing patterns, and so on and on, can be stored as an accumulated history of the individual. Changes in those bio metrics can be observed by the system and its machine learning AI central (or distributed) apparatus which uses those individualized and personalized bio patterns alongside similar pattern changes analyzed and learned from other individuals data (alongside self-reported symptoms) are used to refine AI prediction. The large amount of data continuously fed into the machine learning apparatus performs much better than predictions made based on non-conclusive medical opinions formed through a quick visit to a doctor office, for example. When symptoms of infectious diseases share common symptoms with something like the normal seasonal flu, the machine learning apparatus is capable of recognizing more subtle personalized bio differences and community learned bio differences collected by the system from multiple individuals. The machine learning algorithm can quickly identify new infectious trends and differentiated parameters (especially if they can be combined with self-reporting symptoms that can complement that bio sensed metrics) using the application.

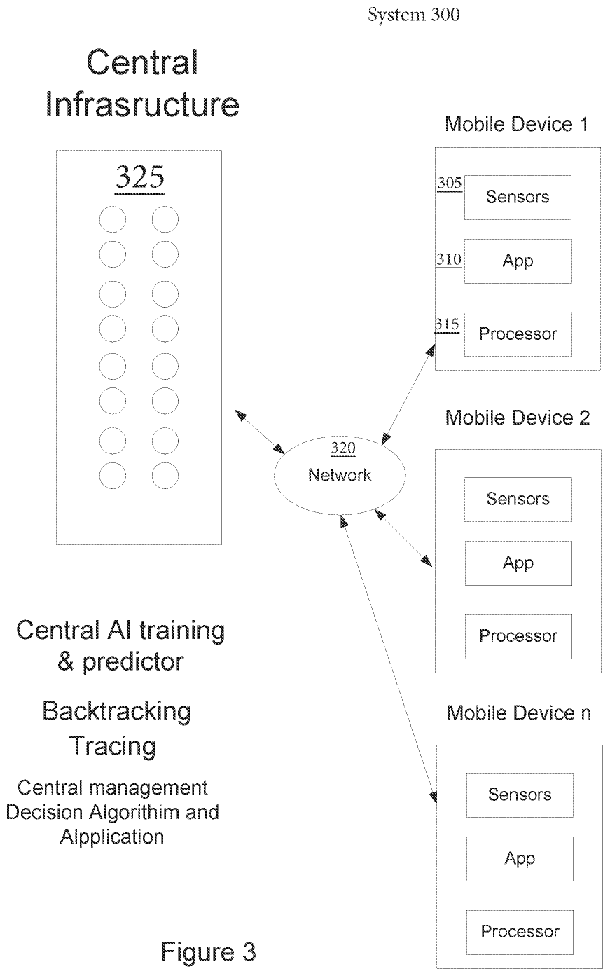

FIG. 3 depicts an exemplary computer system 300 including a central AI device 325 and multiple mobile devices in communication over a wireless network for tracking, tracing, and predicting infections according to embodiments of the present invention. System 300 includes mobile device 1, mobile device 2, and mobile device n communicating with a central infrastructure apparatus 325 over wireless network 320. Fewer or more mobile device can be used in accordance with embodiments. Mobile device 1 includes sensors 305, a processor 315, and a software application 310 executed by processor 315. The software application 310 can capture data using sensors 305 and the data can be securely, privately, and anonymously stored in mobile device 1. Further, Mobile device 1 can transmit the data captured by the sensors (e.g., bio data, biometric features, location data, time data, proximity data, etc.) to central AI device 325. According to some embodiments, the data is transferred as anonymous data points that cannot be associated with a specific individual. Mobile device 1 can further verify an identity of a device holder (e.g., using sensors 305) and can display a notification to the device holder if they are predicted to be exposed to an infection, or are determined to have been exposed to an infection in the past. Mobile device 1 can also compare new sensor data with previously captured data to detect a difference in the captured data that may indicate illness or exposure.

According to some embodiments, the AI machine learning system (e.g., central AI device 325) can use baseline medical symptom diagnoses knowledge as a starting point for training, and will quickly be able to arrive at much better AI based diagnoses and prediction as more points of data are collected and the feedback of infection/illness status is confirmed. A central system (or globally-connected but distributed system) can enable the learning obtained in a remote location (such as another country) to quickly be used and applied in another country that may be just starting to experience the pandemic.

According to some embodiments, in addition to or in place of an AI machine learning system that is used to predict the potential illness or its infectious nature, a remote medical expert can evaluate the data authenticated by and obtained by the device. A tele-medicine system for remote authenticated diagnoses and medical management of illnesses can be provided in this way in case of inability or unavailability of physical examination. This can be the case either in pandemic situation or even in establishing a remote telemedicine trusted environment that can also utilizes an aiding AI machine learning system that uses the sensors of mobile devices alongside identifying management application that can use previously described sensors and mechanisms to combine symptom analysis with identity and biological data authentication, and linking captured through face, eye image, thumbprint, voice, identity recognition, and biological data authenticated collection and analysis. Some embodiments obtain observational studies about the impact of certain physical behavior (e.g., running or exercise), or the effect of consumption of certain supplements or foods on the infection/illness progress or lack of it through self-reporting mechanisms and directed surveys analyzed through the system where infection/illness was predicted by the AI apparatus but did not materialize. These studies and surveys are enabled by embodiments of the present invention. Traditional medical environment rely on physical medical examination, and people who were exposed to the infection/illness but developed light symptoms and not severe ones are never captured in the traditional medical system. Thus all the potential observational studies on the effectiveness or unique features of those individuals who escaped the materialization of the infection or sickness are lost in the traditional medical system but can be studied and evaluated using embodiments of the present invention with great confidence that the study parameters are authentically linked and authenticated to the biological data belonging to the same individual for whom similar past biological data were collected.

The combining identity recognition through thumbprint, face image/video and voice audio and the associated bio sensed metrics (body temperature, eyes/face image-extracted bio-indicators, voice extracted bio indicators e.g., hoarseness, breath patterns, etc., and other metrics) is a unique application of this device that forms a trusted environment where other actions can be based on such trust stemming from the biological data linking and authentication alongside other factors to provide a pandemic management system that utilizes a mobile device, including its bio and other sensors, within a system that combines the device, an AI machine learning apparatus, and a managing application. The machine learning application can be utilized to predict and manage actions based on the AI predicted safe or fail outcome to request or give priority to those AI predicted device users for physical testing, or to allow entry to an office, business, country, airplane, etc., and this can be executed in a distributed manner with high predictability in real time rather than requiring every person to go through actual testing indiscriminately. For example, when an individual is about to enter a class or board mass transit, the application can predict and issue a likely safe code bar on the personal mobile device (similar to how a boarding base can be presented on mobile phones) that can be automatically or manually processed at the entrance of the such class room or mass transit based on the bio history collected and analyzed by the machine learning AI apparatus.

The individual and personal identification scheme described above can maintain privacy as each individual history is secured by the personal mobile device and is available to the system as anonymous data points. The recorded data is only used when the person chooses to use it to obtain access or an entry permit during a pandemic, or when the authorities request history back tracking and tracing to avoid imminent danger to other individuals when a person is confirmed as infectious so that all others who were collocated with him in space and time are alerted.

Another embodiment of the bio linking and authentication system and methods described herein can be employed without limitation using other devices other than a smart phone or wearable device such as an electronically constructed smart test/diagnosis strips, where for example a blood drop from a fingertip can be combined with a test strip that takes an image of the fingerprint to authenticate that the blood sample was obtained from same finger by merging the finger print touch surface alongside the needle and the blood drop sampling surface. This provides a trusted environment in which the identity of the user (in this case the person giving blood drop sample) is linked using his biometric finger print with the biological data being analyzed (blood drop) which enables remote (e.g., at home) trusted testing without the need to physically seek testing in medical facilities with physical personal identification (e.g., using a driver license or similar ID). Embodiments of the present invention provide trusted, remotely managed, and anonymously carried out procedures where identity is usually required to document the medical results/diagnosis or other similar medical data. Furthermore, this approach allows a system including a smart phone and wearable devices to be linked to multiple other bio linked and authenticated data collection smart devices (e.g., a smart blood testing strip) to provide a trusted, secured, and anonymous environment to manage pandemics and establish a telemedicine system, or carry out trusted medical/biological studies where the biological data, tests, diagnosis, etc., are uniquely linked and authenticated to be for the same individuals for which these biological data/tests/diagnosis belongs. Future data/tests/diagnosis can be based on the stored data, and trust-based (identity-based) actions can rely on the data while at the same time maintaining the real personal identity of the user concealed and hidden.

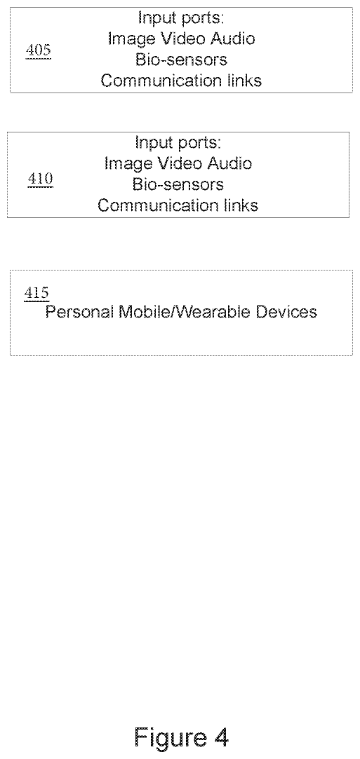

FIG. 4 depicts exemplary smart devices 405, 410, and 415 for tracking, tracing, and predicting infections according to embodiments of the present invention. Smart device 405 is a smartphone that includes image, video, and audio input ports, bio-sensors (e.g., fingerprint scanner, blood pressure monitor, thermometer, pulse monitor, etc.). Smart device 410 is a personal computer that includes image, video, and audio input ports, bio-sensors (e.g., fingerprint scanner, blood pressure monitor, etc.). Smart device 415 is a personal wearable device, such as a smartwatch or band.

According to some embodiments, all personal bio and location information is kept anonymous, secure and confidential. A token is generated and used to isolate (e.g., encrypt) the reference to the device or user identity, and an authentication scheme of the device is executed (e.g., using thumbprint and face/voice recognition). In contrast to traditional schemes, the same biometrics data (e.g., image, thumbprint, voice) that is used for authenticating the user of the device are also used to collect biological data for diagnosis analysis and prediction by AI devices. The authenticated identity recognized using the face/eye image, thumbprint, or voice, and the bio extracted features, such as thumb temperature, face/eye medical image-extracted characteristics color, texture, slimness, paleness, voice hoarseness/breath patterns etc., obtained from the same data and capture sources or device ports are linked together to form an authenticated user collected biological data to insure bio identity-verified and bio analyzed diagnosis prediction, safe status generation and colocation history warnings.

The linking and verifying algorithms described herein can also hide or keep secret the personal identity (e.g., name and address, phone number, device ID) using the verified bio identity to encrypt the biological data and the safe/unsafe prediction generation by the AI apparatus, as well as safe/unsafe status display and used by the device and any status-reading machine. The bio authenticated and bio linked identity can also be used to generate a unique token/key used to facilitate/encrypt data storage and transfer for an anonymous communication, recording, storing, and exchanging of biological data, safe status/diagnosis prediction, location history and bio identity. Other known mechanisms to complement the privacy, anonymity and security provided can be also used alongside the described scheme. For example, some embodiments use advanced encryption, virtual private networks (VPN) and other secure and private communication and storage to improve the privacy, anonymity and security of user information.

According to some embodiments, to maintain privacy and confidentiality of individuals, all recorded data can only be accessed by consensus of each individual. For example, all information on the central apparatus can be encrypted and only used for anonymous tracking and training of the AI/machine learning apparatus. The only time that the device uses individual information is to issue a potential safe pass (similar to boarding pass/entry pass/attendance permit) for boarding mass transit, entering a business complex, or attending a class. It also can notify the individual of the need to do a testing because the AI machine predicted a possible infection in progress as determined by tracing and back tracking. The individuals (or in limited cases Medical authorities) are notified of such a possibility but none of the individual information such as location history is shared with anyone else. The location/proximity information can be used to determine a likelihood of possible infection so that testing/self-isolation or notification of potential hazard from the infection/illness. Beside using the location/proximity information for back tracking and tracing for the purpose of notifying of a possible encountered infection, it also can be used to similarly notify the individual of a potential current health hazard if he happens to be present at/or is planning a trip to a particular location where there is potential existence of predicted infections as indicated by the central apparatus. Anonymous information predicted/confirmed by the central apparatus can be used to create a map view of presence/concentration of such non safe infectious cases to be shown on a map such as the area/city/country map.

Some embodiments of the present invention generate circles of collocation which define, for example, members of the same family who stay in the same house. The circles of colocation help to notify individuals if any of the members of their circle have potential colocation or predicted non safe status. In this way the family members can avoid meeting or potentially coming back to the house by receiving this warning ahead of time. Moreover, thane individual device connected to the central apparatus can notify its holder that there is currently a potentially non-safe situation due to potential infection hazard (unsafe colocation or unsafe environment) so that the individual can know to leave or depart from the location he is currently present at. Unsafe environment means there is currently potentially another individual/individuals who are predicted/confirmed by the central apparatus to be in unsafe status. Unsafe past colocation means that individual might have been in this location in the past (within a time frame defined by the survivability of the infection organism outside the body on surfaces or in the air). These exemplary warnings (warning for future, present, and past colocations) are completely managed within the system and directly from the central apparatus to the individual mobile smart device with no violation of privacy or confidentiality and keeping all information anonymous.

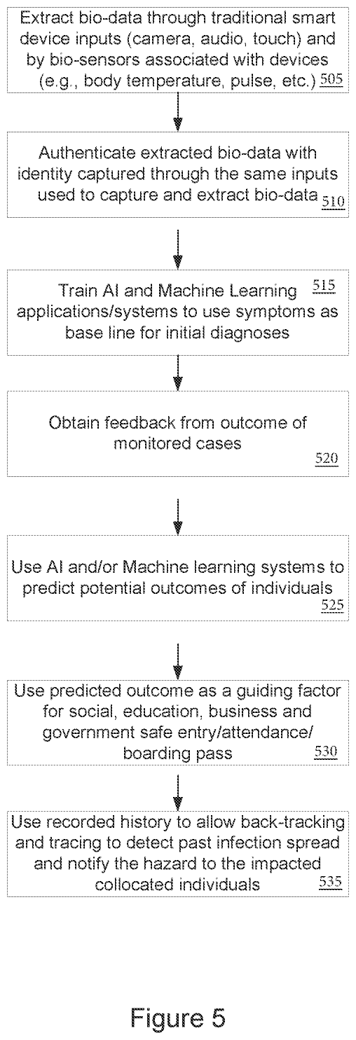

FIG. 5 depicts an exemplary sequence of computer implemented steps for automatically tracking and tracing an infection using a smart device and a machine learning algorithm according to embodiments of the present invention. The steps can be realized as memory stored instructions that can be executed by a computer system.

At step 505, bio data is extracted via a smart device input and bio sensors associated with the device.

At step 510, the bio data is authenticated based on an identity captured using the same input as used to capture the extracted bio data.

At step 515, an AI and machine learning application uses symptoms as a base line for initial diagnosis.

At step 520, feedback is obtained from monitored cases.

At step 525, the AI and machine learning application predicts potential outcomes of individuals.

At step 530, the predicted outcome is used as a guide for social, education, business, and government safe entry/attendance/boarding pass.

At step 535, the recorded history is used to back-track and trace data to detect past infection spread and notify impacted individuals of the hazard.

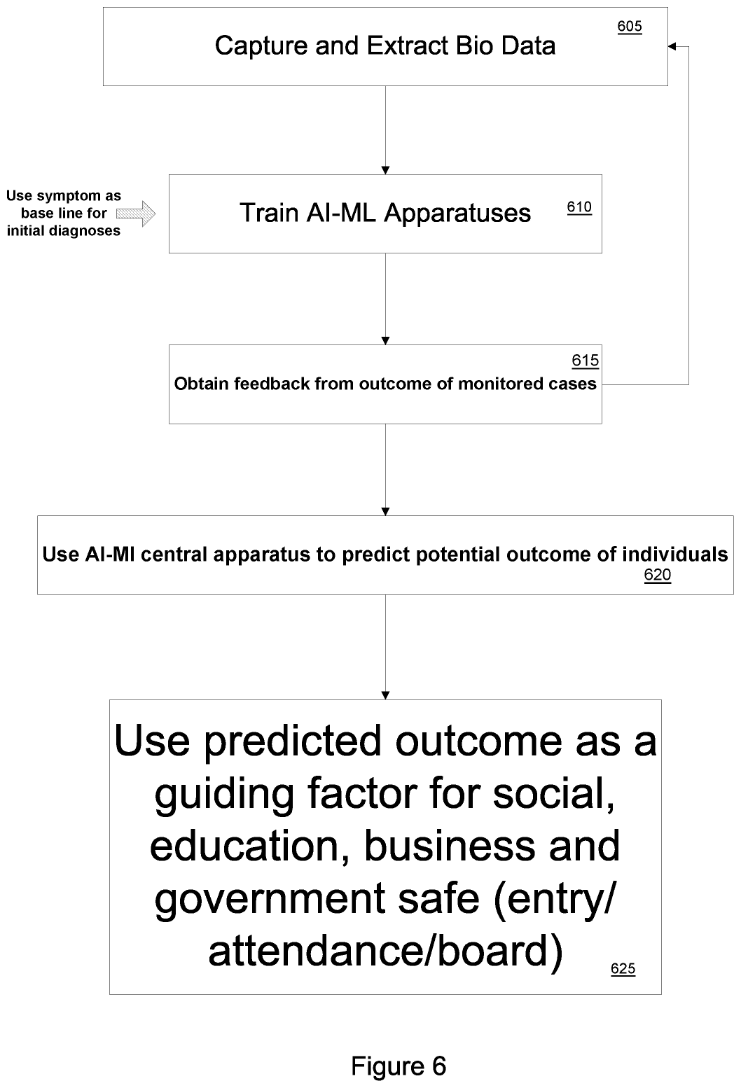

FIG. 6 depicts an exemplary sequence of computer implemented steps for automatically tracking and tracing an infection using a smart device and a machine learning algorithm according to embodiments of the present invention.

At step 605, bio data is extracted.

At step 610, an AI and machine learning application is trained using symptoms as a base line for initial diagnosis.

At step 615, feedback is obtained from monitored cases.

At step 620, the AI and machine learning application predicts potential outcomes of individuals.

At step 625, the predicted outcome is used as a guide for social, education, business, and government safe entry/attendance/boarding pass.

Embodiments of the present invention are thus described. While the present invention has been described in particular embodiments, it should be appreciated that the present invention should not be construed as limited by such embodiments, but rather construed according to the following claims.

What is claimed is:

1. A method of securely tracking potential infections of a contagious disease, the method comprising:
verifying an identity of a first user using a first sensor of a mobile computer device;
capturing a biometric feature of the first user using the first sensor of the mobile computer device;
training an artificial intelligence (AI) machine learning apparatus using baseline symptoms associated with a contagious disease; and
the AI machine learning apparatus predicting a likelihood of potential infection of the contagious disease of the first user according to the biometric feature.

2. The method as described in claim 1, wherein said training comprises:
monitoring a plurality of cases associated with the contagious disease;
obtaining feedback at the AI machine learning apparatus based on outcomes of the plurality of cases; and
training the AI machine learning apparatus based on the outcomes.

3. The method as described in claim 1, further comprising issuing a pass to the first user based on the likelihood of potential infection that was predicted.

4. The method as described in claim 1, further comprising capturing location data of the mobile computer device.

5. The method as described in claim 4, wherein the location data is associated with a time window.

6. The method as described in claim 5, further comprising determining that a second user was located in close proximity to the first user during the time window.

7. The method as described in claim 6, further comprising sending a notification to the second user indicating a potential exposure to the contagious disease.

8. The method as described in claim 5, further comprising sending a notification to the first user indicating a potential exposure to the contagious disease.

9. The method as described in claim 1, wherein the mobile computer device stores the biometric feature privately and anonymously.

10. The method as described in claim 1, wherein the first user is associated with a circle of proximity, and further comprising notifying users within the circle of proximity of a potential exposure within the circle of proximity.

11. A system for securely tracking potential exposure to a contagious disease, the system comprising:
an artificial intelligence (AI) machine learning apparatus; and
a first smart device operable to engage in communication with the AI machine learning apparatus and further operable to execute a software application that performs a first method comprising:
verifying an identity of a first user using a first sensor of the first smart device; and
capturing and storing a biometric feature of the first user using the first sensor of the first smart device,
wherein the AI machine learning apparatus is operable to execute a software application that performs a second method comprising:
performing machine learning using baseline symptoms associated with a contagious disease; and
predicting a likelihood of potential infection of the contagious disease of the first user according to the biometric feature.

12. The system as described in claim 11, further comprising a second smart device, wherein the second method further comprises sending a notification to the second smart device indicating a potential exposure to the contagious disease.

13. The system as described in claim 11, wherein the performing machine learning of the second method further comprises:
monitoring a plurality of cases associated with the contagious disease;
obtaining feedback at the AI machine learning apparatus based on outcomes of the plurality of cases; and
training the AI machine learning apparatus based on the outcomes.

14. The system as described in claim 11, further comprising issuing a pass to the first user based on the likelihood of potential infection.

15. The system as described in claim 11, wherein the first method further comprises capturing location data of the first mobile device.

16. The system as described in claim 15, wherein the location data is associated with a time window.

17. The system as described in claim 15, further comprising sending a notification to the first user indicating a potential exposure to the contagious disease.

18. The system as described in claim 11, wherein the first smart device stores the biometric data privately and anonymously.

19. A smart device comprising:
a finger scanner input device;
a camera input device;
an audio input device; and
a processor in electronic communication with the finger scanner input device, the camera input device, and the audio input device, wherein the processor is operable to execute instructions to automatically perform a method comprising:
authenticating a user using at least one of the input devices;
capturing biometric data of the finger scanner input device, the camera input device, and the audio input device;
transmitting biometric data anonymously to an artificial intelligence (AI) machine learning apparatus; and
receiving a predicted outcome for the user from the AI machine learning apparatus generated according to the biometric data.

20. The smart device of claim 19, wherein the AI machine learning apparatus is trained using the biometric data, and wherein the biometric data comprises at least one of: an image of a user's face captured by the camera input device; audio of the user's voice captured by the audio input device; and temperature data captured by the finger scanner input device.

21. The smart device of claim 19, wherein the method further comprises displaying an indication that the predicted outcome is a negative outcome, and wherein the indication maintains the anonymity of the user.
